(19) United States
(12) Patent Application Publication
(10) Pub. No.: US 2016/0207994 A1
(43) Pub. Date: Jul. 21, 2016

(54) MODULATORS OF IL-12 AND/OR IL-23 FOR THE PREVENTION OR TREATMENT OF ALZHEIMER'S DISEASE

(71) Applicants: UNIVERSITAT ZURICH, Zurich (CH); CHARITE UNIVERSITATSMEDIZIN BERLIN, Berlin (DE)

(72) Inventors: Burkhard Becher, Maur (CH); Frank Heppner, Berlin (DE)

(73) Assignees: UNIVERSITAT ZURICH, Zurich (CH); CHARITE UNIVERSITATSMEDIZIN BERLIN, Berlin (DE)

(21) Appl. No.: 15/000,063

(22) Filed: Jan. 19, 2016

Related U.S. Application Data
(63) Continuation of application No. 13/978,149, filed on Jul. 3, 2013, now abandoned.

(30) Foreign Application Priority Data
Jan. 4, 2011 (EP) 11150090.6
Apr. 12, 2011 (EP) 11161991.2

(51) Int. Cl. C07K 16/24; G01N 33/50; A61K 31/5377; A61K 31/202; A61K 38/17; C12N 15/113

(52) U.S. Cl. CPC C07K 16/244; A61K 38/1793; C12N 15/1136; A61K 31/5377; A61K 31/202; G01N 33/5058; C07K 2317/92; A61K 2039/505

(57) ABSTRACT

The invention provides antibody against p40, IL-12 or IL-23 for the prevention or treatment of Alzheimer's disease. It further provides ligands to the pair of interleukin 12 or 23 and its specific receptor, specifically an antibody, an antibody fragment, an antibody-like-molecule, for the prevention or treatment of Alzheimer's disease. Similarly, siRNA, antisense or transcription factor modulators of gene expression of p19, p35, p40, IL-12R-β1, IL-12R-β2, and/or IL-23R for the prevention or treatment of Alzheimer's disease are provided.

### CROSS-REFERENCE TO RELATED APPLICATIONS

This is a continuation of co-pending U.S. patent application Ser. No. 13/978,149 filed Jul. 3, 2013, which is the U.S. National Stage of International Application No. PCT/EP2012/050066, filed Jan. 4, 2012, which was published in English under PCT Article 21(2), and which in turn claims the benefit of European Patent Application No. 11161991.2, filed Apr. 12, 2011, and European Patent Application No. 11150090.6, filed Jan. 4, 2011.

### FIELD OF THE INVENTION

The present invention relates to polypeptide or nucleic acid inhibitors or modulators of IL-12 or IL-23, or receptors thereof, for the prevention and treatment of Alzheimer's disease. In particular, the invention relates to anti-p40-antibodies for the prevention and treatment of Alzheimer's disease.

### BACKGROUND

Interleukin-12 (IL-12) and Interleukin-23 (IL-23) are heterodimeric cytokines consisting of a common subunit called p40 (interleukin 12 subunit beta, Uniprot ID P29460), which either pairs with p35 (interleukin 12 subunit alpha, Uniprot ID P29459) for IL-12 or with p19 (interleukin 23 subunit alpha, Uniprot ID Q9NPF7) for IL-23 (Oppmann et al. Immunity 13:715-725 (2000)). IL-12 polarizes the so-called T helper cell (TH) 1 subset, while IL-23 is an essential factor for the expansion and survival of a novel effector cell subset coined TH17. In addition, both factors are increasingly understood to also impact on a number of innate leukocytes.

IL-12 binds to the IL-12 receptor (IL-12R), which is a heterodimeric receptor formed by IL-12R-β1 (Uniprot ID P42701) and IL-12R-β2 (Uniprot ID: Q99665). IL-12R-β2 is found predominantly on activated T and NK cells and is stimulated by cytokines that promote TH1 cell development. IL-23 binds to and signals through its heterodimeric receptor complex composed of IL-12Rβ1 and IL-23R (Uniprot ID: Q5VWK5) subunits (Parham, C. et al. J Immunol 168:5699-5708 (2002)). Whereas IL-12Rβ1 is also part of the IL-12 receptor, IL-23R is unique to the IL-23 receptor complex.

Alzheimer's disease (AD) is the most common form of dementia. No cure exists for AD. The inventors are not aware of any published data proving or indicating a positive effect of modulators of IL-12 and/or IL-23 in fighting Alzheimer's disease.

### SUMMARY OF THE INVENTION

The central feature of the instant invention is the surprising finding that an anti-p40-antibody exerts a strong effect on the formation of AD in a relevant rodent model. Without wishing to be bound by any theory, all data generated in the exploration of this invention indicate that inhibiting the interaction of the interleukin subunit p40, in the context of its interleukin heterodimers IL12 or IL23, with its physiological receptors, leads to the observed effect.

### BRIEF DESCRIPTION OF THE FIGURES

FIGS. 1a-1c: Upregulation of IL-12 and/or IL-23 in brains of Alzheimer's APPPS1 mice: Gene expression analysis of pooled APPPS1 mouse brain cells compared to C57BL/6 control mice at 120d of age exhibit an upregulation of inflammatory cytokines (IL-23p19, IL-12/IL-23p40 and TNFα) in the myeloid CD11 b⁺ CD45⁺ cell fraction (FIG. 1b), while only a slight upregulation of IL-12 p35, but not of IL-12/IL-23p40 and of IL-23p19 was noted in the non-myeloid CD11 b⁻ CD45⁻ cell fraction (FIG. 1a). Treatment of isolated postnatal microglial cell cultures with synthetic Aβ₁₋₄₂ peptides (22.5 μg/ml) induced an almost 6-fold upregulation of IL-12/IL-23p40 after 24 h when compared to unstimulated controls (FIG. 1c).

FIGS. 2a-d: Genetic deletion of IL-12 and/or IL-23 subunits reduces Aβ-plaque load in Alzheimer's APPPS1 mice

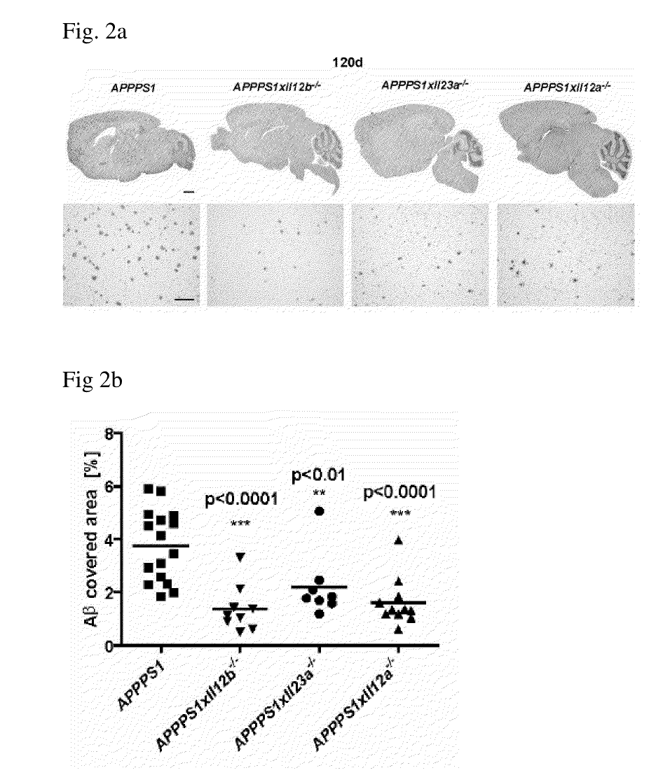

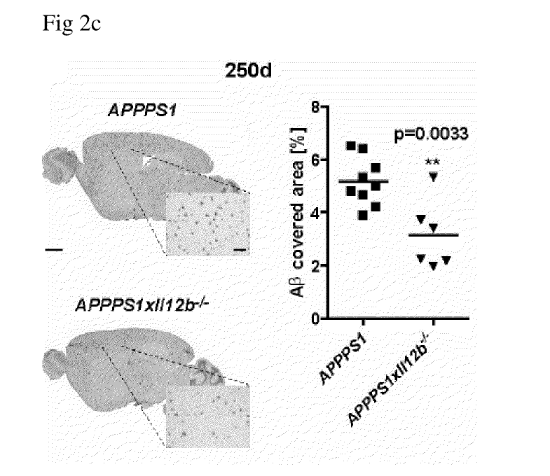

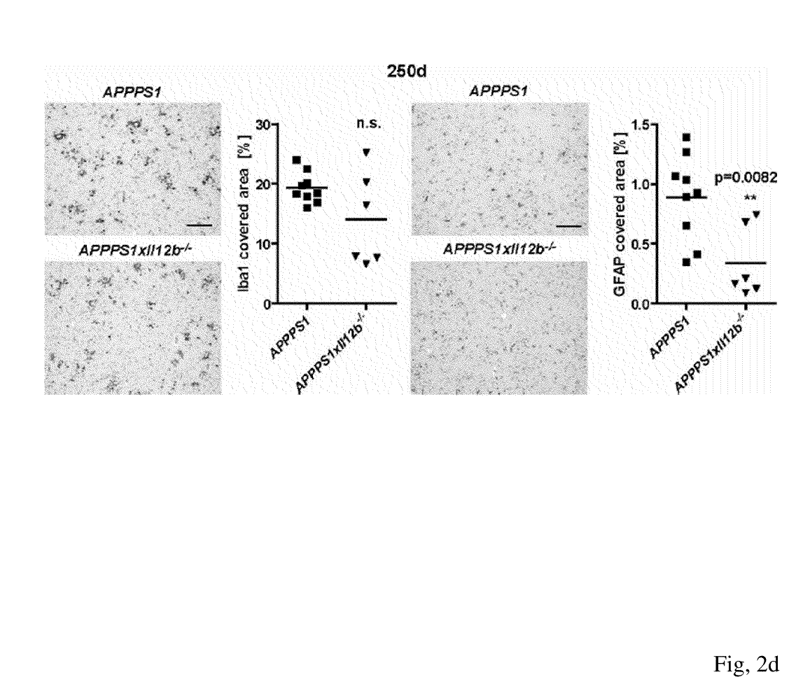

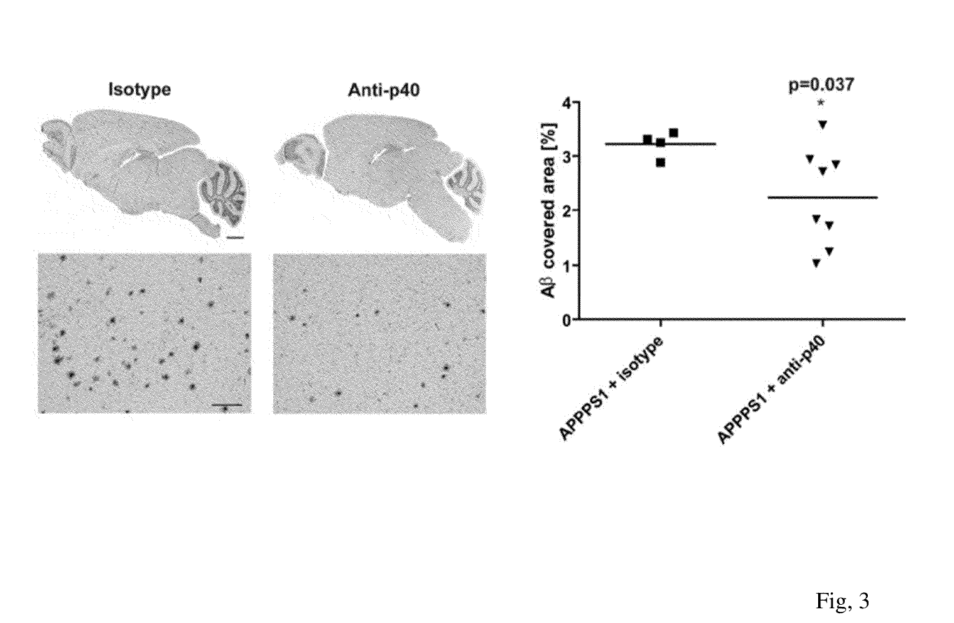

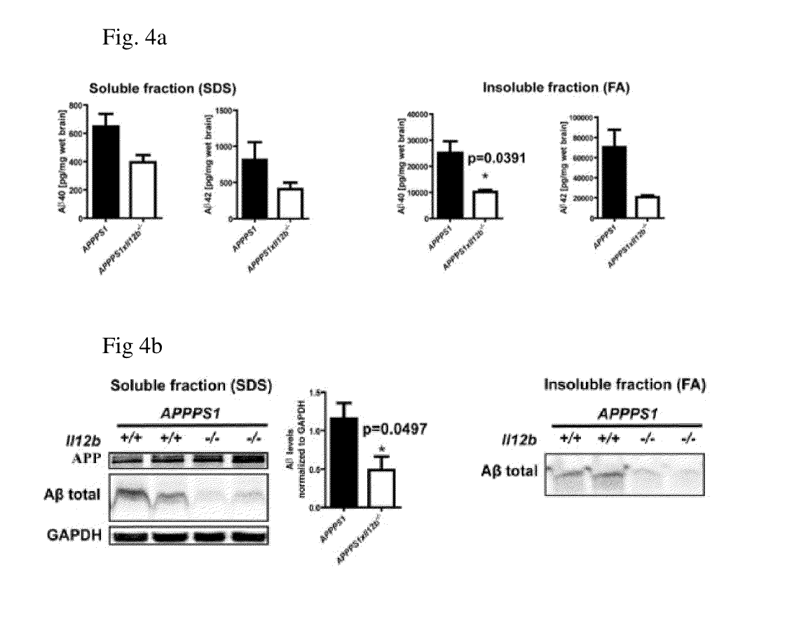

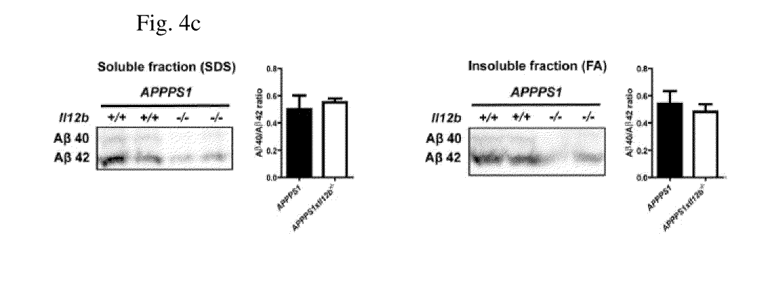

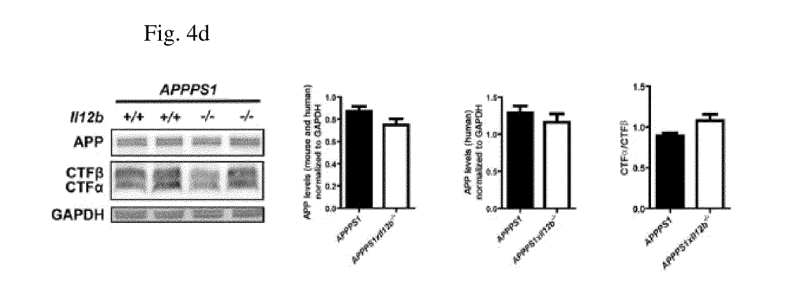

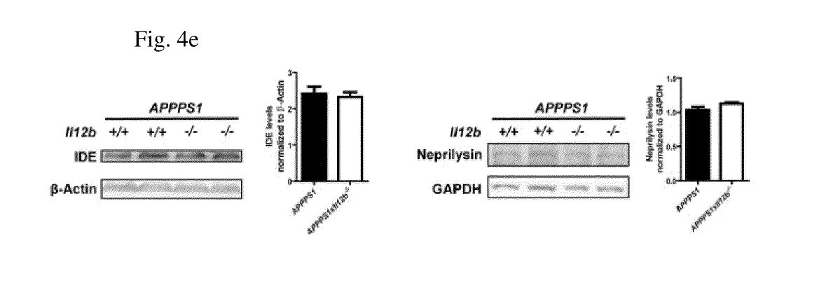

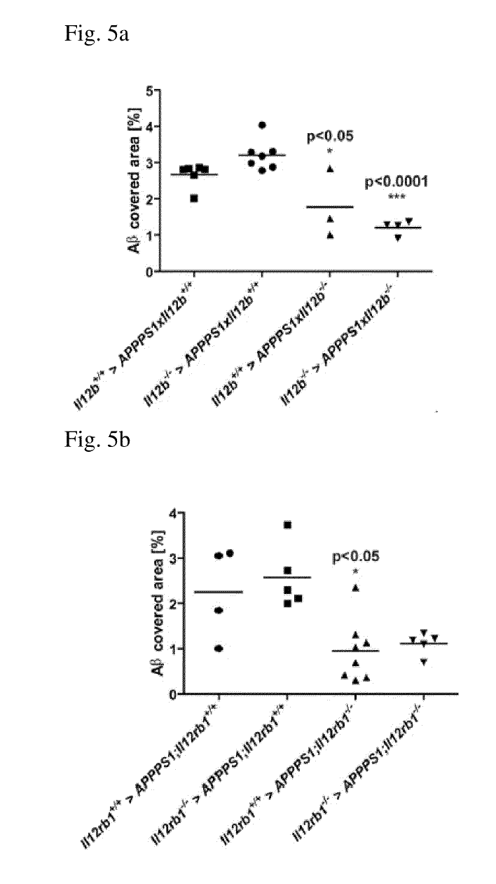

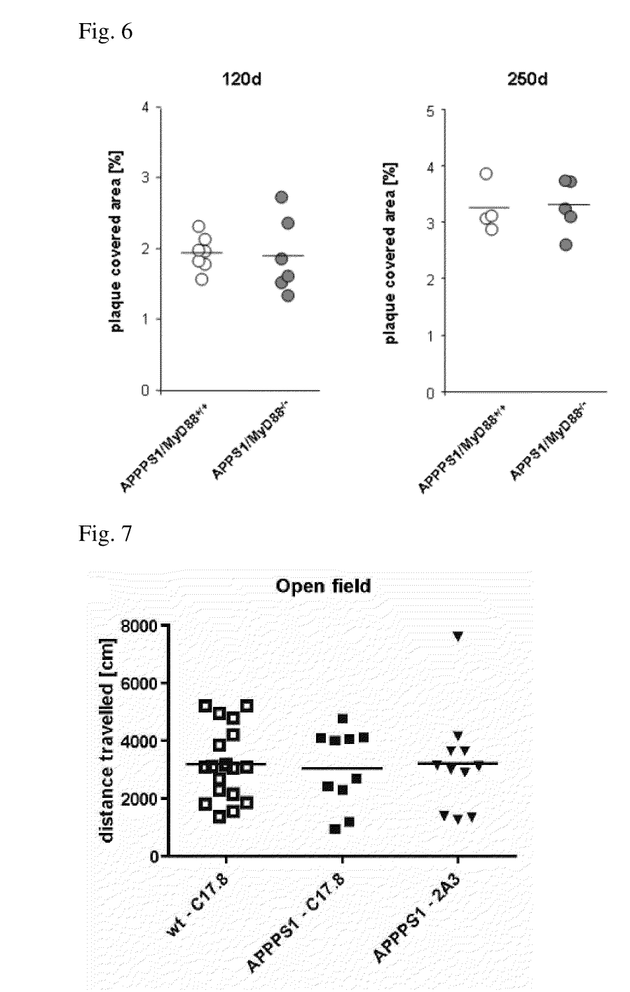

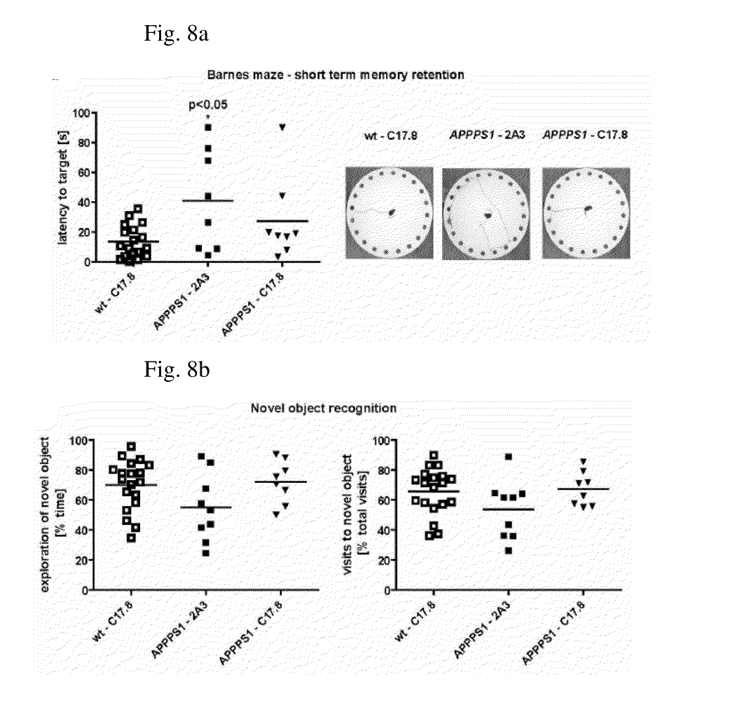

(FIG. 2a) Aβ burden in cytokine-deficient APPPS1 mice was assessed by immunohistochemical staining using the β-amyloid reactive antibody 4G8 (upper row: low magnification pictures, scalebar 500 μm; lower row: higher magnification images used for morphometric quantification, scalebar 200 μm). As shown and reported by many others, Alzheimer's APPPS1 mice have a robust Aβ-plaque load as early as 120 d of age without significant inter- or intraindividual variability. (FIG. 2b) Morphometric analysis of β-amyloid covered area in APPPS1 mice lacking the IL-12 and/or IL-23 subunits p40 (II12b^{-/-}, n=9), p19 (II23a^{-/-}, n=8) or p35 (II12a^{-/-}, n=11) at 120d of age, compared to control Alzheimer's APPPS1 mice with functional IL-12 and/or IL-23 signaling (n=16). Morphometric analyses were performed by measuring the immunstained area of 3 cortical regions representing the frontal, parietal and occipital cortex (each 1.4 mm²) on every 50^{th} systematically sampled 6-μm-thick brain section. The stereomorphologic analysis of 27 defined regions/per mouse was performed with the aid of the Cell D software (Olympus, Tokyo, Japan) using the color filter and phase analysis tool. (FIG. 2c) Analysis of β-amyloid plaque load at 250d of age in APPPS1 mice compared to APPPS1×II12b^{-/-} mice (left: low magnification picture, scalebar 500 μm; insert: high magnification image used for morphometric quantification, scalebar 200 μm; right: morphometric quantification of APPPS1 (n=9) and APPPS1×II12b^{-/-} (n=6) mice. (FIG. 2d) Staining for and quantification of microglia/macrophages (left: histological panel and morphometric quantification using Iba1 antibody, scalebar 100 μm) and astroglia (right: histological panel and morphometric quantification using GFAP antibody, scalebar 100 μm) at 250d of age in APPPS1 (n=9) and APPPPS1×II12b^{-/-} (n=6) animals. Each dot represents the mean of the morphometrically assessed Aβ plaque load, Iba1- or GFAP-covered area of one mouse.

FIG. 3: Peripheral administration of anti-p40 antibodies to Alzheimer's APPPS1 mice results in a robust and statistically significant reduction of Aβ plaques

Alzheimer's APPPS1 mice were treated with anti-p40 antibodies (C17.8; n=8) or isotype control antibodies (2A3; n=4). A single bolus intraperitoneal (i.p.) injection of 0.5 mg of anti-mouse II-12/23p40 antibody (clone C17.8, rat IgG2a) or of the respective rat isotype control (clone 2A3) at 4 weeks of age was followed by biweekly i.p. injections of 0.25 mg of the respective antibodies until the end of the experiment (120 days of age). Representative overview and high magnification images of plaque burden in treated animals (4G8 staining; scale bars as in FIG. 2). Morphometric analyses of the Aβ burden (performed as described in FIG. 2) revealed an overall profound and statistically significant reduction of Aβ-burden exclusively in the anti-p40 treatment group, which was pronounced in at least 4 out of 8 APPPS1 mice. Each dot represents the mean of the morphometrically assessed Aβ plaque load of one mouse.

FIGS. 4a-e: Genetic deletion of IL-12/IL-23 p40 reduces Aβ-plaque burden in APPPS1 mice without altering amyloid precursor protein (APP) processing

Biochemical analysis was performed on homogenates of cerebral hemispheres of APPPS1 and APPPS1×II12b^{-/-} mice at 250d of age. (FIG. 4a) Quantitative analysis of Aβ40 and Aβ42 levels in brain homogenates using the MesoScale ELISA system in the soluble (SDS, left panel) and insoluble (FA, right panel) fraction. (FIG. 4b) Levels of Aβ total and human (transgenic) APP in the soluble (SDS) fraction (left panels) were assessed by Western blot analysis using the 6E10 antibody for detection and densitometric analysis thereof. Aβ total levels were analyzed in a similar fashion in the insoluble (FA) fraction (right panel). (FIG. 4c) Aβ40 and Aβ42 species in the soluble (left panel) and insoluble (right panel) fraction were separated using a specialized SDS-Urea gel system, detected with the 6E10 antibody and quantified by densitometric quantification. (FIG. 4d) Expression levels of endogenous murine and transgenic human APP and major C-terminal cleavage products of APP (CTFα and CTF) were assessed by Western blot analysis (left panel) using the APPct antibody and quantified by densitometric scanning (right panel, which also includes quantification of the Western blot shown in (b)). (FIG. 4e) Expression levels of the major Aβ degrading enzymes insulin degrading enzyme (IDE; left panel) and neprilysin (right panel) were analyzed by Western blot analysis using specific antibodies and densitometric quantification. Representative Mesoscale results, Western blot images and results of densitometric quantification of at least 3 independent experiments are shown (n=3-7 mice per group). GAPDH or β-Actin were used as internal control for quantification. Error bars depict s.e.m.

FIGS. 5a and 5b: Deficiency of IL-12/IL-23 p40 or IL-12 signalling in the radioresistant compartment is sufficient to reduce Aβ-plaque load in Alzheimer's APPPS1 mice

Aβ plaque load assessed as in FIG. 2 at 120d of age 11 weeks after reconstitution (FIG. 5a) APPPS1 or APPPS1×II12b^{-/-} mice were lethally irradiated at 6 weeks of age and reconstituted with bone marrow isolated from C57BL/6 mice (wt) or II12b^{-/-} mice of the same age (n=3-7 animals per experimental group). Shown is one out of two representative experiments. (FIG. 5b) APPPS1 or APPPS1×II12rb1^{-/-} mice were lethally irradiated at 6 weeks of age and reconstituted with bone marrow from C57BL/6 mice or II12rb1^{-/-} mice (n=4-8 animals per experimental group).

FIG. 6: Manipulating innate immunity does not change Aβ-plaque load in Alzheimer's APPPS1 mice: to prove the specificity of reducing the Alzheimer's Aβ-plaque load by targeting IL-12 and/or IL-23 signalling, mice lacking the key adaptor molecule MyD88 required for innate immune functions (and irrelevant for IL-12 and/or IL-23 signalling) were crossed to APPPS1 mice. APPPS1/MyD88^{-/-} mice at 120 or 250 days of age revealed no change in Aβ burden when compared to APPPS1/MyD88^{+/+} mice (a, b). Each dot represents the mean of the morphometrically assessed plaque load of one mouse. The morphometrical analysis has been performed as described in the legend to FIG. 2.

FIG. 7: Behavioral analysis of aged mice upon icy antibody delivery

Aged wt and APPPS1 mice (230-240 days of age) were subjected to behavioral analyses after 6 weeks of intracerebroventricular (icy) anti-p40 or isotype control antibody treatment (a) Assessment of general locomotor activity in the Open-Field arena quantified as distance travelled [cm]. One symbol represents data of one mouse.

FIGS. 8a and 8b: Intracerebroventricular delivery of anti-p40 antibodies to aged APPPS1 mice results in amelioration of behavioral deficits

Aged wt or APPPS1 mice were treated with anti-p40 antibodies (C17.8) or isotype control antibodies (2A3). Antibodies were delivered intracerebroventricularly (icy) using an Alzet miniosmotic pump at a concentration of 2 mg/ml and a flowrate of 0.24 μl/hr starting at 190 days of age. After 6 weeks of treatment, mice underwent behavioral tests. (FIG. 8a) Representative pathway tracking pictures (right panel) and quantification of latency to reach target (left panel) for Barnes maze—short term memory retention trial. (FIG. 8b) Quantification of % time spent exploring novel object (left panel) and % visits to new object (right panel) for novel object recognition. One way Anova p-value=0.085. Each symbol represents data of one mouse (wt+anti-p40 n=18; APPPS1+ isotype n=8; APPPS1+ anti-p40 n=8).

### SEQUENCE LISTING

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named 95083_303_3001_seqlist.txt, about 4.5 KB, which is incorporated by reference herein.

### DETAILED DESCRIPTION

According to a first aspect of the invention, an antibody against p40, interleukin 12 or interleukin 23, is provided for the prevention or therapy of Alzheimer's disease. According to one embodiment, an anti-IL-12 antibody is provided for treatment or prevention of Alzheimer's disease. According to one embodiment, an anti-p-40 antibody is provided for treatment or prevention of Alzheimer's disease. According to one embodiment, an anti-IL-23 antibody is provided for treatment or prevention of Alzheimer's disease.

According to an alternative of this first aspect of the invention, an inhibitor of the interaction of IL-12 with its IL-12-receptor is provided for the prevention or treatment of Alzheimer's disease. Alternatively, an inhibitor of IL-23-IL-23 receptor interaction is provided for prevention or treatment of AD. An inhibition of the IL-12-IL-12 receptor interaction or the IL-23-IL-23-receptor interaction results in reduced inflammatory reaction and ultimately in a strong and significant reduction in the Aβ-plaque load. Aβ-plaque load is a pathognomonic indicator of AD.

Such inhibitor according to the first aspect of the invention may be an antibody, an antibody fragment or an antibody-like-molecule, each directed against and binding to any of the members of the interleukin-interleukin-receptor pair of IL-12 or IL-23. The subunits of IL-12 or IL-23, namely p19, p35 or p40, may be the target of such antibody; similarly, the subunits of the IL12 receptor or of the IL23 receptor, as are IL-12R-β1, IL-12R-β2 and IL-23R may be the target. The dissociation constant of such antibody, antibody fragment or antibody-like-molecule from its target typically is in the sub-micromolar range, for example below 10⁻⁷ mold, below 10⁻⁸ mold or even <10⁻⁹ mold.

Suitable inhibitors according to the first aspect of the invention may be developed by evolutive methods such as phage display, ribosome display or SELEX, wherein polypeptide or oligonucleotides are selected due to their binding affinity to a target of interest. Additionally, the binding affinity of an identified inhibitor may be improved by cycles of evolution of the amino acid sequence or nucleotide sequence, and selection of the evolved inhibitors may be effected based on the required affinity.

One example of an antibody fragment according to the first aspect of the invention is a Fab fragment. A Fab fragment is the antigen-binding fragment of an antibody. An example for an antibody-like molecule is a repeat protein, such as a “designed ankyrin repeat protein” (Molecular Partners, Zürich). Both antibody fragments and antibody-like molecules effect the modulation or inhibition of IL-12's biological activity or IL-23's biological activity by binding to IL-12 and/or IL-23, by depleting the respective cytokine from the extracellular space or by blocking the interaction between the cytokine and its receptor. Antibodies against IL-12 and/or IL-23 are known in the art and include the well characterized antibodies ustekinumab (CAS 815610-63-0; a.k.a. Stelara; commercialized by Janssen Biotech) and briakinumab (CAS No. 339308-60-0; developed by Abbott Laboratories).

According to a preferred embodiment, an antibody for prevention or therapy of AD binds to the IL-12 receptor and/or the IL-23 receptor in the region where IL-12 and/or IL-23 bind within the physiological context of ligand-receptor interaction, but without inducing IL-12 and/or IL-23-downstream physiological effects.

According to a preferred embodiment, an antibody for prevention or therapy of AD is an anti-IL23R antibody as described in WO/2008/106134 (“ENGINEERED ANTI-IL-23R-ANTIBODIES”; Schering Corporation) or in WO/2010/027767 (“ENGINEERED ANTI-IL-23R ANTIBODIES”, Schering Corporation), or an anti-p40-antibody as described in WO/2010/017598 (“ANTI-IL-12/IL-23 ANTIBODIES”, Arana Therapeutics Ltd).

An antibody against IL-12 and/or IL-23 or its receptors can be raised by methods known in the art, for example by immunization of knockout mice using the virus-like particle system, or by injection of recombinant protein in knockout mice.

Additionally, proteins and protein analogues that bind to IL-12 or IL-23, or to their respective receptors, may be employed to practice the invention. Synthetic proteins or protein analogues that mimic the variable region scFv of binding and/or neutralizing antibodies are one example; antibodies that mimic a binding pocket for IL-12 and/or IL-23 on its receptors are another example.

Alternatively, the inhibitor according to the first aspect of the invention may be an oligopeptide of 6 to 30 amino acids or a nucleic acid aptamer molecule of no more than 75 nucleotides in length, said oligopeptide or nucleic acid aptamer molecule being capable to bind to a member of the group comprised of IL-12, IL-23, the ID 2 receptor, the IL23 receptor, p19, p35, p40, IL-12R-β1, IL-12R-β2, and IL-23R. The binding of said oligopeptide or nucleic acid aptamer to said member of the IL-12 or IL-23 signalling group is characterized by a dissociation constant of 10⁻⁸ mold or smaller.

According to a third alternative, the inhibitor according to the first aspect of the invention may be a soluble polypeptide comprising a sequence of at least 30 amino acid residues in length taken from the amino acid sequence of p40, p35, p19, IL-12R-β1, IL-12R-β2, or IL-23R. Said sequence of at least 30 amino acids binds to the IL-12R-β1, IL-12R-β2, or IL-23R (if the sequence is a part of the interleukin polypeptide) or to the interleukine polypeptide (if the sequence is part of the receptor polypeptide), either binding taking place without eliciting the biological effect of native interleukin-interleukin-receptor interaction. Optionally said sequence of at least 30 amino acids is linked to an Fc antibody domain. The rationale is to provide a soluble decoy for either of the interleukin—interleukin receptor pair, wherein the decoy outcompetes native interleukin signalling.

According to one preferred embodiment of this alternative, the soluble polypeptide may be an extracellular domain of the IL-12-receptor or the IL-23-receptor fused to a constant fragment Fc of an antibody, for example an immunoglobulin G. One example of such preferred embodiment is an immunofusion protein consisting of IL12-p40 and a hybrid Fc domain, as commercially available from Genexine (Korea). Said hybrid Fc domain is a hybrid human Fc domain of (i) IgG1, IgG2 or IgG4 or (ii) IgG4 and IgD (US 2008300188 A1)

According to a second aspect of the invention, a modulator of gene expression of p19, p35, p40, IL-12R-β1, IL-12R-β2, and/or IL-23R is provided for the prevention or treatment of Alzheimer's disease. Such modulator may be a single-stranded or double-stranded interfering ribonucleic acid oligomer or a precursor thereof, comprising a sequence tract complementary to an mRNA molecule encoding any of p19, p35, p40, IL-12R-β1, IL-12R-β2, and/or IL-23R, for degradation of said mRNA molecule.

The art of silencing or “knocking down” genes, by degradation of mRNA or other effects, is well known. A multitude of mechanisms of action for, and definitions of, such RNA-driven interference with gene expression has been discovered and adapted to practical use during the last decade. Examples of technologies identified are siRNA, miRNA, shRNA, shmiRNA, or dsRNA. A comprehensive overview of this field can be found in Perrimon et al, Cold Spring Harbour Perspectives in Biology, 2010, 2, a003640.

Likewise, the modulator may be an expression vector comprising an RNA polymerase promoter sequence operable in a mammalian cell, and an expressed sequence encoding said interfering ribonucleic acid oligomer or a precursor thereof. Such expression vector leads to production of an interfering RNA within the cell. Methods for making and using such expression vectors are known in the art.

Alternatively, the modulator according to the second aspect of the invention may be a single-stranded or double-stranded antisense ribonucleic or deoxyribonucleic acid, comprising sequences complementary to a regulatory region of a gene encoding a member of the group comprised of p19, p35, p40, IL-12R-β1, IL-12R-β2, and IL-23R.

Such antisense molecules may be 12-50 nucleotides in length, preferably 18-30 nucleotides, and have a sequence comprised in an exon or intron of any of the subunits of IL-12 or IL-23. Moreover, antisense molecules comprising a sequence forming part of the promoter sequence regulating transcription of any of the IL-12 or IL-23 subunits, and binding to one of the strands of the promoter region, may be used. Finally, antisense molecules binding in the 3′ UTR non-translated regions of any of the subunits of IL-12 and/or IL-23 are contemplated.

According to a third aspect of the invention 6-morpholino-N-((E)-m-tolylmethyleneamino)-2-(2-(2-pyridyl)ethoxy)pyrimidin-4-amine (I)

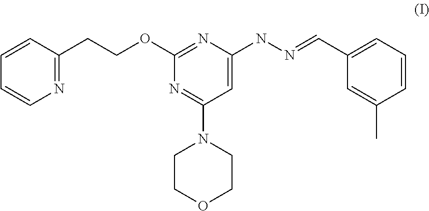 [SMILES: Cc1cccc(/C=N/Nc2cc(N3CCOCC3)nc(OCCc3ccccn3)n2)c1]

(Apilimod, Synta Pharmaceuticals STA 5326) is provided for the prevention or therapy of Alzheimer's disease. This compound is an orally administered small molecule inhibitor of IL12 and IL-23 that inhibits the intracellular translocation of a transcription factor driving p40 transcription (Keino et al., Arthritis Research and Therapy 10, R122 (2008)).

According to a fourth aspect of the invention the polypeptides Ustekinumab (Janssen-Cilag), Briakinumab (Abbott Laboratories), CEP-37248 (Cephalon, Inc), FM 202 (Femta Pharmaceuticals), LY2525623 (Eli Lilly), MP196 (TcL Pharma), anti-IL23-Antibodies as described in WO/2008/103432 (“ENGINEERED ANTI-IL-23P19 ANTIBODIES”; Schering-Plough) and GP04 (Genexine Co., Ltd.) are provided for the prevention and treatment of Alzheimer's disease. Said polypeptides are antibodies directed against IL-12, IL-23, or their receptors except for GP04, which is an immunofusion protein and acts as an IL-23 receptor antagonist.

According to a fifth aspect of the invention, a resolvin compound is provided for the prevention or treatment of Alzheimer's disease. Resolvin compounds are lipid mediators generated through oxidation of omega-3 fatty acids eicosapentaenoic acid (EPA) or docosahexaenoic acid (DHA). They have anti-inflammatory properties (Serhan et al. J. Exp. Med. 196, (8):1025-37, 2002). Resolvin E1, a member of the resolvins, dramatically decreases the proinflammatory gene expression, i. a. IL-12/p40 (Arita. et al. PNAS 21:7671-7676, 2005). Likewise Resolvin D2, another member of the resolvins, drastically reduces the levels of proinflammatory cytokines, namely IL-6, IL-1β IL-23 and TNF-a (Spite et al., Nature, 461(7268), 1287-1291, 2009).

Preferred embodiments according to this fifth aspect of the invention are listed below:
Resolvin E1 (5S, 12R, 18R)-5, 12, 18-trihydroxyicosa-6, 8, 10, 14, 16-pentaenoic acid), enantiomers and racemates thereof:

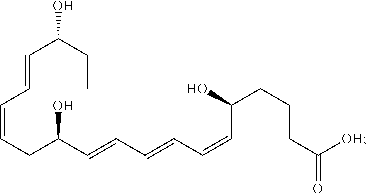 [SMILES: CC[C@@H](O)/C=C/C=C\C[C@@H](O)/C=C/C=C/C=C\[C@@H](O)CCCC(=O)O]

Resolvin E2 (5S, 18R)-5, 18-dihydroxyicosa-6, 8, 11, 14, 16-pentaenoic acid), enantiomers and racemates thereof:

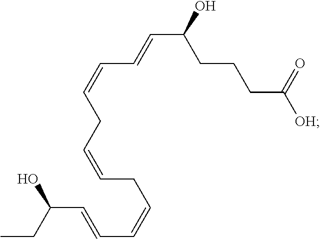 [SMILES: CC[C@@H](O)/C=C/C=C\C/C=C\C/C=C\C=C\[C@@H](O)CCCC(=O)O]

RX-10045 (Resolvyx Pharmaceuticals, a synthetic resolvin analogue formulated for topical application to treat eye disease);
Resolvin D1 (7S, 8R, 17R)-7, 8, 17-trihydroxydocosa-4, 9, 11, 13, 15, 19-hexaenoic acid), enantiomers and racemates thereof:

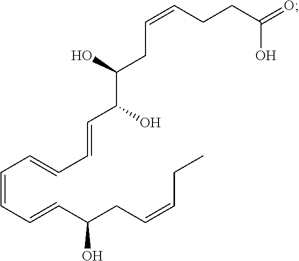 [SMILES: CC/C=C\C[C@@H](O)/C=C/C=C\C=C\C=C\[C@@H](O)[C@@H](O)C/C=C\CCC(=O)O]

Resolvin D2 (7S, 17S)-7, 16, 17-trihydroxydocosa-4, 8, 10, 12, 14, 19-hexaenoic acid), enantiomers and racemates thereof:

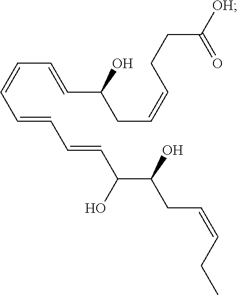 [SMILES: CC/C=C\C[C@H](O)C(O)/C=C/C=C/C=C\C=C\[C@@H](O)C/C=C\CCC(=O)O]

Resolvin D3 (4S, 17S)-4, 11, 17-trihydroxydocosa-5, 7, 9, 13, 15, 19-hexaenoic acid), enantiomers and racemates thereof:

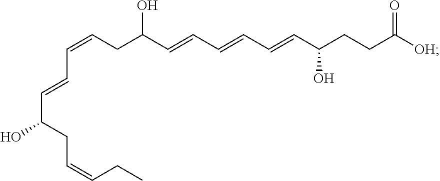 [SMILES: CC/C=C\C[C@H](O)/C=C/C=C\CC(O)/C=C/C=C/C=C/[C@@H](O)CCC(=O)O]

Resolvin D4 (4S, 17S)-4, 5, 17-trihydroxydocosa-6, 8, 10, 13, 15, 19-hexaenoic acid), enantiomers and racemates thereof:

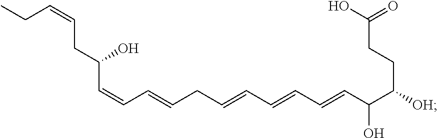 [SMILES: CC/C=C\C[C@H](O)/C=C\C=C\C/C=C/C=C/C=C/C(O)[C@@H](O)CCC(=O)O]

Resolvin D5 (7S, 17S)-7, 17-dihydroxydocosa-5, 8, 10, 13, 15, 19-hexaenoic acid, enantiomers and racemates thereof:

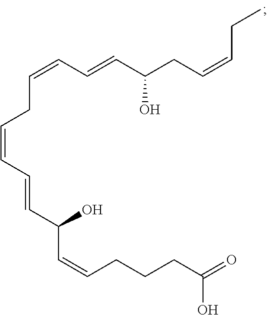 [SMILES: CC/C=C\C[C@H](O)/C=C/C=C\C/C=C\C=C\[C@@H](O)/C=C\CCCC(=O)O]

Resolvin D6 (4S, 17S)-4,17-dihydroxydocosa-5, 7, 10, 13, 15, 19-hexaenoic acid, enantiomers and racemates thereof:

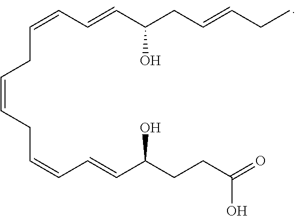 [SMILES: CC/C=C/C[C@H](O)/C=C/C=C\C/C=C\C/C=C\C=C\[C@@H](O)CCC(=O)O]

According to another aspect of the invention a peptide for prevention or treatment of Alzheimer's disease is provided, wherein the peptide is between 5 and 25 amino acid residues in length comprising an amino acid sequence selected from the group comprised of TEEEQQYL, TEEAQQYL, TAAEQQYL, TAAEQQYL, TAAAQQYL, EEEQQYL, EEQQYL, EQQYL, AEEQQYL, TEEEQQYL, TEEEQQ, TEEEQ, IEEE; TEEEQAYL, TEEEAAYL, MEESKQLQL, MAESKQLQL, MAASKQLQL, ESKQLQL, MEESKQLQI, MEESKQL, MEESKQ, MEESQQLQI, EESKQLQL, VQAANALGMEESKQLQLHLDDLVL, LVLDDLHLQLQKSEEMGLANAAQV, LPDEVTCV and KKYLVWVQ.

These peptides correspond to flexible regions of the IL-23 receptor and derivatives thereof. The peptides antagonize the biological activity of the IL-23R by inhibition of oligomerisation of the extracellular domain due to their promotion or stabilization of particular conformations. Methods of manufacturing the peptides and further information are described in WO/2009/007849.

According to preferred embodiment of the preceding aspect, at least one of the amino acid residues is a D-amino acid residue.

According to another aspect of the invention, a pharmaceutical composition for the prevention or treatment Alzheimer's disease is provided, comprising an inhibitor or modulator according to any of the above claims.

Similarly, a dosage form for the prevention or treatment of Alzheimer's disease is provided, comprising an inhibitor or modulator according to one of the above aspects of the invention. Dosage forms may be for enteral administration, such as nasal, buccal, rectal, transdermal or, especially, oral administration, or as an inhalation form or suppository. Oral administration of proteins is described in WO2007029238A2 (Kidron, Oramed Pharmaceuticals, Jerusalem, IL). Alternatively, parenteral administration may be used, such as subcutaneous, intravenous, intrahepatic or intramuscular injection forms. Optionally, a pharmaceutically acceptable carrier and/or excipient may be present.

Said pharmaceutical compositions comprise from approximately 1% to approximately 95% active ingredient, preferably from approximately 20% to approximately 90% active ingredient.

An injection form of a said pharmaceutical composition is preferred. According to a preferred embodiment, solutions of an inhibitor or modulator according to any of the above aspects of the invention can be made up shortly before use as an injection form. Similarly, suspensions or dispersions, especially isotonic aqueous solutions, dispersions or suspensions may be employed.

Said pharmaceutical compositions for oral administration also include hard capsules consisting of gelatin, and also soft, sealed capsules consisting of gelatin and a plasticizer, such as glycerol or sorbitol. The capsules may contain the active ingredient in the form of granules, or dissolved or suspended in suitable liquid excipients, such as in oils.

According to one embodiment, a dosage form for injection for prevention or treatment of Alzheimer's disease comprises 5 mg, 10 mg, 15 mg, 20 mg, 25 mg, 30 mg, 35 mg, 40 mg, 45 mg, 50 mg, 60 mg or 75 mg of the antibody ustekinumab or briakinumab. According to one embodiment, a dosage regime for prevention or treatment of Alzheimer's disease comprises one injection form of 5 mg, 10 mg, 15 mg, 20 mg, 25 mg, 30 mg, 35 mg, 40 mg, 45 mg, 50 mg, 60 mg or 75 mg of the antibody ustekinumab or briakinumab every two, three or four weeks over a period of two, three, four, five or six months.

Transdermal/intraperitoneal and intravenous applications of said pharmaceutical compositions are also considered, for example using a transdermal patch, which allows administration over an extended period of time, e.g. from one to twenty days.

Intravenous or subcutaneous application of said pharmaceutical compositions are preferred modes of practicing the invention. Intracranial delivery is particularly preferred.

The dosage of the active ingredient depends upon the species, its age, weight, and individual condition, the individual pharmacokinetic data, the mode of administration, and whether the administration is for prophylactic or therapeutic purposes. In the case of an individual having a bodyweight of about 70 kg the daily dose administered is from approximately with 0.1 mg/kg to approximately 1000 mg, preferably from approximately 0.5 mg to approximately 100 mg/kg, of a modulator of the IL-12 and/or IL-23.

A pharmaceutical composition comprising an inhibitor or a modulator according to one of the above aspects can be administered alone or in combination with one or more other therapeutic agents. A combination therapy may take the form of fixed combinations of said pharmaceutical composition and one or more other therapeutic agents known in the prevention or treatment of Alzheimer's disease. Administration may be staggered; alternatively the drugs may be given independently of one another, or in the form of a fixed combination.

Possible combination partners considered are memantine (Lundbeck), donepezil (Eisai), galantamine (Janssen) and rivastigmine (Novartis).

Also within the scope of the present invention is a method for the prevention or treatment of Alzheimer's disease, comprising the use of an inhibitor or modulator according to one of the above aspects of the invention.

Similarly, a method for the manufacture of a medicament for the prevention or treatment of Alzheimer's disease is provided, comprising administering an inhibitor or modulator according to one of the above aspects of the invention to a patient in need thereof. Medicaments according to the invention are manufactured by methods known in the art, especially by conventional mixing, coating, granulating, dissolving or lyophilizing.

According to yet another aspect of the invention, a method is provided for identifying a compound suitable for the prevention or treatment of Alzheimer's disease, comprising the steps of
incubating cells in the presence of IL-12 and/or IL-23, and in the presence of a compound that is to be examined with regard to its suitability as an Alzheimer drug. The cells used for such method of identification comprise a receptor for IL-12 and/or a receptor for IL-23 and exhibit a detectable response to interaction of IL12 with the IL12-receptor and/or interaction of IL23 with IL23-receptor.
measuring the cell's response to interaction of interleukin with its corresponding receptor, and
comparing the response in presence of said compound to a standard response.

Cells suitable for this assay express receptors for IL-12 or IL-23 and corresponding signal transduction pathways downstream of said receptors. An incubation of such cells with IL-12 or IL-23 may result in a physiological response. According to a preferred aspect of the above aspect, the method may be a cell-based bioassay, wherein splenocytes (phagocytic cells of the spleen) are incubated in a tissue culture medium in the presence of II-12 or IL-23. IL-12 and IL-23 stimulate the release of interferon gamma and IL-22, respectively, into the medium. The release of interferon gamma and IL-22 may be measured in the absence and the presence of compounds. Varying concentrations of said compound can be tested in a high-throughput 384 well system.

Modulators of IL-12 and/or IL-23 activity are identified by contacting IL-12 and/or IL-23 (or its receptors) with a candidate compound. A control assay with the corresponding IL-12 and/or IL-23 (or its receptors) in the absence of the candidate compound is run in parallel. A decrease in activity in the presence of the candidate compound compared to the level in the absence of the compound indicates that the candidate compound is a IL-12 and/or IL-23 modulator.

The action of p40-containing heterodimers IL-12 and/or IL-23 can be modulated by administration of antibodies or antibody fragments directed against IL-12 and/or IL-23 or their subunits p40, p35 and p19, or fusion proteins consisting of IL-12R and/or IL-23R extracellular domains fused to the Fc portion of antibodies (i.e. cytokine traps) as well as by administration of small molecules that interfere with the binding of ligands to the receptors of IL-12 or IL-23. The production of IL-12 and/or IL-23 may be modulated by using siRNA in vitro but also by directly suppressing the promoter activity of genes of IL-12 and/or IL-23 subunits with small molecules or suppressors of the transcription factors involved in the transcription of the respective genes. The action of IL-12 and/or IL-23 can be inhibited by IL-12 and/or IL-23 receptor modulators. Additionally, targeting of IL-12 and/or IL-23 can be achieved by the administration of neutralizing antibodies or antibody fragments to IL-12 and/or IL-23 or by proteins, protein analogs or small synthetic compounds which bind IL-12 and/or IL-23 and thereby prevent its binding to the IL-12 and/or IL-23 receptor, or bind to the IL-12 and/or IL-23 receptor. A further way to prevent binding to the IL-12 and/or IL-23 receptor is to use soluble IL-12 and/or IL-23 receptor molecules or fragments thereof.

Most preferred modulators according to the invention are:
ustekinumab (Centocor), a high affinity, fully human antibody that targets IL-12 and IL-23 (via p40 subunit);
briakinumab (Abbott Laboratories), a fully human monoclonal antibody that targets IL-12 and IL-23 (via p40 subunit);
CEP-37248 (Cephalon, Inc.), a humanized antibody targeting the interleukin 12/23 pathway;
FM202 (Femta Pharmaceuticals), a monoclonal antibody directed against the common p40 subunit of IL-12 and IL-23;
LY2525623 (Eli Lilly & Co.), an IL-23 antibody;
MP196 (TcL Pharma SAS), a monoclonal antibody against IL-23;
Anti-IL-23p19 antibody (Schering-Plough), an antibody targeting the p19 subunit of IL-23;
ATI003 (Bristol-Myers Squibb Company), an IL-12 antagonist;
ADC-1012 (Alligator Bioscience AB), a selective IL-23 antagonist;
APG2305 (Allostera Pharma, Inc.), an oral IL-23 receptor inhibitor;
RX10001 (Resolvyx Pharmaceuticals, Inc.), an IL-23 inhibitor that contains resolvinE1, an endogenous lipid mediator derived from omega-3 docosapentaenoic acid, and RX10045, an eikosapentanoic acid derivative;
GXP04 (Genexine Co., Ltd.), an immunofusion protein consisting of IL-12p40 and hybrid Fc, acting as IL-23 receptor antagonist
anti-IL23R antibodies as described in WO/2008/106134 (“ENGINEERED ANTI-IL-23R-ANTIBODIES”; Schering Corporation) and in WO/2010/027767 (“ENGINEERED ANTI-IL-23R ANTIBODIES”, Schering Corporation)
anti-p40-antibodies as described in WO/2010/017598 (“ANTI-IL-12/IL-23 ANTIBODIES”, Arana Therapeutics Ltd).

### DETAILED DESCRIPTION OF THE INVENTION

The herein reported invention provides evidence that manipulation of cytokines of the adaptive immune system, namely of IL-12 and/or IL-23 or its receptors, can impact Aβ-plaque burden in Alzheimer's disease (AD). The importance of IL-12 and/or IL-23 or its receptors in the pathogenesis of AD was highlighted by the following aspects:
AD brains display an inflammatory component characterized by the presence of proinflammatory cytokines particularly in response to AD β-amyloid (Aβ), which is a pathognomonic indicator of AD.
It was found that myeloid cells, namely microglia and macrophages, typically surrounding Aβ-plaques and known to be a major source of inflammatory cytokines in the brain, showed an robust upregulation e.g. of IL-12/IL-23 p40 and IL-23 p19 in Alzheimer's APPPS1 mice when compared to age-matched wild-type controls.
Genetic ablation of IL-12 and/or IL-23 or its receptors (e.g. of p40, p19, p35 and iI12rb1) resulted in a consistent, strong and significant reduction in the Aβ-plaque load of Alzheimer's APPPS1 mice at various time points investigated (120 and 250 days of age)
In order to demonstrate the feasibility of manipulating IL-12 and/or IL-23 or its receptors for treating AD, blocking anti-p40 antibodies were injected peripherally (intraperitoneally) into Alzheimer's APPPS1 mice and induced a significant reduction of Aβ burden.
In behavioral tests, a significant deficit in short term memory retention was substantially ameliorated by direct intracerebroventricular delivery of anti-p40 antibodies using mini-osmotic pumps.

Manipulation of IL-12 and/or IL-23 signalling thus provides a new therapeutic strategy to fight AD, even when applied outside the brain.

The invention is further illustrated by the following figures and examples, from which further embodiments and advantages may be derived.

### EXAMPLES

### Example 1

### Cytokine Levels in Sorted Brain Cells of APPPS1 Mice

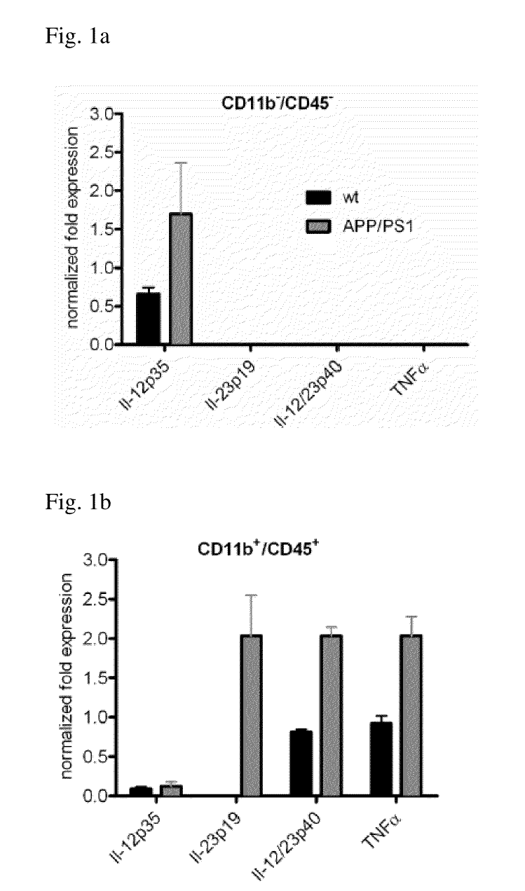

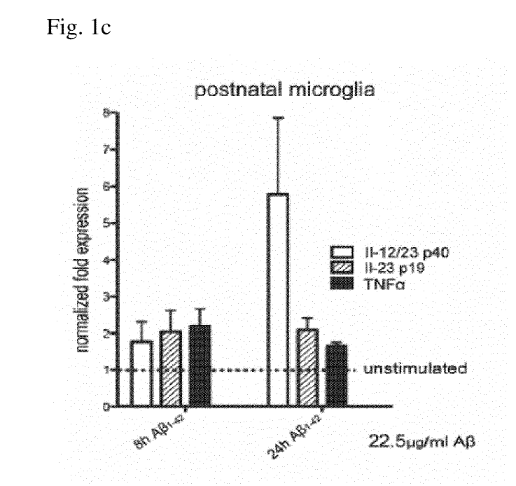

Alzheimer's disease (AD) pathology displays an inflammatory component characterized by the presence of proinflammatory cytokines and reactive oxygen species particularly in response to AD β-amyloid. Since both in human AD samples as well as in transgenic AD mice, microglia and macrophages surrounding Aβ-plaques are reported to be a major source of inflammatory cytokines, we FACS sorted myeloid brain cells of Alzheimer mice in order to better characterize this inflammatory component. Initially, we assessed the expression of IL-12, IL-23 and TNFα in 4-month-old APPPS1 animals and age matched non-transgenic littermates by quantitative PCR of the two cellular fractions obtained (CD45⁺ CD11b⁺ and CD45⁻ CD11b⁻). This analysis revealed roughly a 1.6 fold upregulation of IL-12p35 in the double negative fraction consisting of non-myeloid cells of the central nervous system, while other cytokines assessed could not be detected (FIG. 1a). In contrast, we observed a robust upregulation of IL-12/IL-23p40 and IL-23p19 in the double positive fraction containing microglia and brain macrophages of APPPS1 mice compared to age-matched littermate controls (FIG. 1b). In addition, treatment of isolated postnatal microglial cell cultures with synthetic Aβ₁₋₄₂ peptides (22.5 μg/ml) induced a substantial upregulation of TNFα, IL-23p19 and most prominently of IL-12/IL-23p40, which yielded an almost 6-fold increase over unstimulated microglia control cultures (FIG. 1c). To validate a potential link between IL-12 and IL-23 subunits and Aβ-pathology in APPPS1 transgenic animals—besides their impact in models of CNS autoimmune disease, IL-12/IL-23 have not been described to play a role in neurodegenerative diseases so far—as well as to test, whether inhibition of IL-12 and IL-23 subunits could alter disease progression in AD mice, genetic and applied experiments targeting IL-12 and IL-23 subunits in AD mice were performed.

### Example 2

### Knockout of IL-12/IL-23 Subunits Reduces Aβ-Plaque Load in APPPS1 Mice

To directly study the impact of IL-12 and IL-23 on Aβ-pathology we crossed APPPS1 mice to
(1) p40^{-/-} (genetic name: II12b^{-/-}) mice (Magram, J. et al., Ann N Y Acad Sci 795:60-70 (1996)) that lack the common component of IL-12 and IL-23,
(2) p35^{-/-} (genetic name: II12a^{-/-}) mice (Mattner, F. et al., Eur J Immunol 26:1553-1559 (1996)) that are deficient in IL-12 and
(3) p19^{-/-} (genetic name: II23a^{-/-}) mice (Cua, D. J et al. Nature 421:744-748 (2003)) that are deficient in IL-23.

At 120d of age all cytokine-deficient APPPS1 mice lacking p40 (APPPS1×II12b^{-/-} mice, n=9), p35 (APPPS1×II12a^{-/-} mice, n=11) and p19 (APPPS1×II23a^{-/-} mice, n=8), showed a substantial reduction in cortical Aβ-plaque load as judged by detailed morphometric analysis (FIGS. 2a and 2b) compared to age-matched APPPS1 control mice. However, this effect was most pronounced in APPPS1 mice lacking p40. Similar results were obtained when analyzed at 250d, where APPPS1/p40^{-/-} (APPPS1×II12b^{-/-}) mice exhibited a significant reduction in Aβ-plaque burden compared to age-matched controls (FIG. 2c). This observation excludes the possibility that cytokine-deficiency merely affects the kinetics of plaque formation. The reduction in plaque burden was accompanied by a reduced astrocytic and microglial reaction in APPPS1/p40^{-/-} (APPPS1×II12b^{-/-}) mice (FIG. 2d).

### Example 3

### Peripheral Administration of Anti-p40 Antibodies Mimics Effect of Genetic p40 Ablation

To further substantiate our findings and to prove that targeting the p40 signalling can be used as a novel interventional approach to treat AD, we pharmacologically blocked p40 signalling in Alzheimer's APPPS1 mice. Alzheimer's APPPS1 mice were treated with anti-p40 antibodies (C17.8; n=8) or isotype control antibodies (2A3; n=4). A single bolus intraperitoneal (i.p.) injection of 0.5 mg of anti-mouse II-12/23p40 antibody (clone C17.8, rat IgG2a) or of the respective rat isotype control (clone 2A3) at 4 weeks of age was followed by biweekly i.p. injections of 0.25 mg of the respective antibodies until the end of the experiment (120 days of age). Despite the fact that APPPS1 mice are a most robust Aβ-plaque carrying AD mouse model, in which, if at all, reduction of the Aβ burden is difficult to achieve, there was an overall profound and statistically significant reduction of Aβ-burden exclusively in the anti-p40 treatment group, which was pronounced in at least 4 out of 8 APPPS1 mice (FIG. 3). All anti-p40 antibody treated APPPS1 mice displayed p40 blocking activity in the serum at the end of the experiment (as assessed by ELISA; data not shown).

### Example 4

### Knockout of IL-12/IL-23 p40 Reduces Aβ-Burden in APPPS1 Mice without Altering APP Processing

The immunohistochemical phenotype of APPPS1/p40^{-/-} (APPPS1×II12b^{-/-}) mice (FIG. 2) was substantiated by subsequent biochemical analyses. FIG. 4 demonstrates a reduction of Aβ40 and Aβ42 species in the soluble (SDS) as well as in the insoluble (FA) fraction in APPPS1/p40^{-/-} (APPPS1×II12b^{-/-}) mice compared to APPPS1 mice harbouring functional p40 subunits at 250d (FIG. 4a-c). This reduction in Aβ-peptides was not accompanied by a shift in the ratio of Aβ40 and Aβ42 arguing against a direct effect of IL-12/IL-23 p40 on the cleavage pattern of γ-secretase that in principle could result in a reduced Aβ burden (FIG. 4c). Also the expression of the amyloid precursor protein (APP) and the ratio of α- and β-CTF, which are direct substrates of γ-secretase, were not changed in APPPS1/p40^{-/-} (APPPS1×II12b^{-/-}) mice compared to APPPS1 mice (FIG. 4d). In addition, levels of IDE and Neprilysin, the two major Aβ-degrading enzymes in the brain, were identical in APPPS1 and APPPS1/p40^{-/-} (APPPS1×II12b^{-/-}) mice (FIG. 4e). These biochemical findings are supported by the lack of differences in the expression of AD-related genes in brains of 250d old APPPS1 compared to APPPS1/p40^{-/-} (APPPS1×II12b^{-/-}) mice, as revealed by a PCR-array study focussing on murine Alzheimer genes (SA biosciences).

### Example 5

### IL-12/IL-23 p40-Deficiency in the Radioresistant Compartment Suffices to Reduce A13-Plaque Load

To dissect the site of p40 action in APPPS1 mice, bone marrow (BM) chimeric mice lacking p40 in the radio-resistant (i.e. the CNS) or in the radio-sensitive, hematopoietic compartment were generated. Flow cytometric analyses of blood for congenic donor markers six weeks after adoptive transfer demonstrated successful reconstitution (data not shown); thereafter, mice were aged and finally analyzed at 120 d for Aβ plaque burden by an immunohistochemical-based morphometric analysis. At the time of termination of the experiments, microglia were not replaced by central nervous system (CNS)-invading donor cells, as expected (data not shown). APPPS1×II12b^{-/-} recipients receiving II12b^{-/-} bone marrow (II12b^{-/-}→APPPS1×II12b^{-/-} retained the previously described p40-dependent phenotype (FIG. 5a), indicating that irradiation and reconstitution per se did not affect cerebral amyloidosis. II12b^{+/+}→APPPS1×II12b^{-/-} mice, which exhibit a loss of the p40 gene in the radio-resistant compartment, displayed a reduction in Aβ plaque load similar to unmanipulated APPPS1×II12b^{-/-} mice (FIG. 5a). Importantly, II12b^{-/-}→APPPS1×II12b^{+/+} chimeric mice showed no decrease in Aβ plaque load when compared to the baseline control (FIG. 5a). This indicates that p40 expression by CNS-resident cells can modulate Aβ plaque load in Alzheimer's APPPS1 mice.

To verify the impact of IL-12 and IL-23 signaling on amyloid burden in AD mice, additional BM chimeric mice lacking IL-12β1 receptor (II12rb1^{-/-}, which is the common subunit shared by both IL-12 and IL-23 receptors and required for conduction of IL-12/23p40-mediated signals (Wu, C. et al., J Immunol 159:1658-1665 (1997)), were created. This approach also allowed determination of the target of CNS-derived and Aβ-mediated p40 expression. Consistent with a role of IL-12/IL-23 signaling in exacerbation of amyloid formation, the absence of IL-12β1 receptor in CNS-resident cells resulted in a drastic reduction of Aβ plaque burden (FIG. 5b). These data shows that lack of IL-12/IL-23 signaling in the radio-resistant compartment of the CNS, i.e. in microglia, suffices to reduce cerebral amyloidosis.

### Example 6

### Manipulating Key Molecules of Innate Immunity does not Change Aβ-Plaque Load in Alzheimer's APPPS1 Mice

To prove the specificity of reducing the Alzheimer's Aβ-plaque load by targeting IL-12 and/or IL-23 signalling, mice lacking the common adaptor molecule MyD88 required for innate immune functions such as Toll-like receptor (TLR) signalling (and irrelevant for IL-12 and/or IL-23 signalling) were crossed to APPPS1 mice. Despite some evidence in the literature for a potential involvement of certain TLRs in Aβ-pathology, APPPS1/MyD88^{-/-} mice exhibited a similar plaque burden at 120 or 250 days of age when compared to hemizygeous APPPS1/MyD88^{+/-} and to APPPS1/MyD88^{+/+} control mice as assessed by a detailed morphometric analysis (FIG. 6). Biochemical analyses of SDS-soluble and insoluble fractions of Aβ40 and Aβ42 using the Mesoscale® electrochemoluminescence system also revealed no major difference between MyD88-deficient and control APPPS1 mice. Thus, yet another change in the immune system of APPPS1 mice, such as the deletion of the MyD88 molecule, does not alter the amount of Aβ-burden in APPPS1 mice, proving the specificity of targeting IL-12 and/or IL-23 signalling in Alzheimer's disease.

### Example 7

### Behavioral Studies

To determine if anti-p40 treatment can also have a functional impact on an already established disease phenotype, we treated aged AD mice with anti-p40 antibodies. Despite early-onset and aggressive Aβ-plaque pathology, APPPS1 mice develop only a mild behavioral phenotype that is not detectable until approximately 8 months of age and restricted to few behavioral tests. In consideration of this point and because our previous findings specify the importance of the radio-resistant central compartment in the mediation of p40-dependent anti-amyloidogenic effects, we delivered neutralizing anti-p40 antibodies for 60 days directly into the lateral ventricle of the brain of 190 day old APPPS1 mice using mini-osmotic pumps. While APPPS1 mice (treated with anti-p40 or isotype control antibodies) did not exhibit any abnormalities in a control test of spontaneous locomotor behavior compared to wt animals (treated with anti-p40 antibodies) (FIG. 7)—a precondition for any locomotor behavior-based cognitive assay-, isotype-treated APPPS1 mice exhibit measurable behavioral deficits in three different cognitive tasks: the contextual fear conditioning paradigm (not shown), Barnes maze (FIG. 8a) and novel object recognition (FIG. 8b). Notably, the significant deficit in short term memory retention observed in APPPS1 mice in the Barnes maze test was substantially ameliorated upon treatment with anti-p40 antibodies (FIG. 8a). Similarly, the obvious deficit shown by APPPS1 mice in the cortical- and hippocampal-dependent novel object recognition task was reversed upon anti-p40 treatment to normal levels comparable to wt littermates (FIG. 8a). Therefore, beyond the observed beneficial effect on the development of Aβ-plaque pathology by targeting p40 signaling, intracerebroventricular (icy) anti-p40 treatment proved to ameliorate the behavioral and cognitive impairment observed in aged APPPS1 mice despite the already well-established Aβ-plaque pathology achieved in this model at this late time point.

We claim:

1. A method for treatment of Alzheimer's disease, comprising:
administering to a patient in need of such treatment a pharmaceutical composition comprising an inhibitor of IL-12-IL-12-receptor interaction, and/or an inhibitor of IL-23-IL-23-receptor interaction, wherein administering the composition treats the Alzheimer's disease.

2. The method of claim 1, wherein the inhibitor comprises an antibody against p40, an antibody against interleukin 12 (IL-12), or an antibody against interleukin 23.

3. The method of claim 1, wherein the inhibitor is ustekinumab or briakinumab, and wherein administering the pharmaceutical composition comprises injecting into the patient 5 mg, 10 mg, 15 mg, 20 mg, 25 mg, 30 mg, 35 mg, 40 mg, 45 mg, 50 mg, 60 mg or 75 mg of the ustekinumab or briakinumab.

4. The method of claim 1, wherein administering the pharmaceutical composition further comprises injecting the ustekinumab or briakinumab every two, three or four weeks over a period of two, three, four, five or six months.

5. The method of claim 1, wherein the inhibitor is selected from the group consisting of: an antibody, an antibody fragment, an antibody-like-molecule, an oligopeptide of 6 to 30 amino acids, and a nucleic acid aptamer molecule of 10 to 75 nucleotides in length, wherein the inhibitor binds with a dissociation constant of 10⁻⁸ mol/l or smaller to a member of the group consisting of: p40, IL-12, IL-23, the IL12 receptor, the IL23 receptor, p19, p35, IL-12R-β1, IL-12R-β2, and IL-23R.

6. The method of claim 1, wherein the inhibitor comprises a soluble polypeptide comprising a contiguous amino acid sequence of at least 30 amino acids from within the amino acid sequence of p40, p35, p19, IL-12R-β1, IL-12R-β2, or IL-23R.

7. The method of claim 1, wherein the inhibitor comprises a modulator of p19, p35, p40, IL-12R-β1, IL-12R-β2, and/or IL-23R expression, and wherein the modulator comprises a single-stranded or double-stranded interfering ribonucleic acid oligomer or precursor thereof, comprising a sequence complementary to an mRNA molecule encoding any of the p19, p35, p40, IL-12R-β1, IL-12R-β2, and/or IL-23R.

8. The method of claim 1, wherein the inhibitor comprises a modulator of p19, p35, p40, IL-12R-β1, IL-12R-β2, and/or IL-23R expression, and wherein the modulator comprises a sequence complementary to a regulatory region of a gene encoding a protein selected from the group consisting of p19, p35, p40, IL-12R-β1, IL-12R-β2, and IL-23R.

9. The method of claim 7, wherein the modulator is produced by an expression vector comprising:
an RNA polymerase promoter sequence operable in a mammalian cell; and
an expressed sequence encoding the interfering ribonucleic acid oligomer or a precursor thereof.

10. The method of claim 1, wherein the inhibitor is 6-morpholino-N-((E)-m-tolylmethyleneamino)-2-(2-(2-pyridyl)ethoxy)pyrimidin-4-amine:

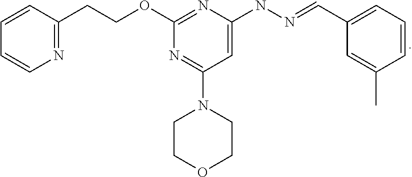 [SMILES: Cc1cccc(/C=N/Nc2cc(N3CCOCC3)nc(OCCc3ccccn3)n2)c1]

11. The method of claim 1, wherein the inhibitor is a polypeptide selected from the group consisting of: ustekinumab, briakinumab, CEP-37248, FM 202, LY2525623, MP196, Anti-IL-23P19 antibodies, anti-IL23R antibodies, anti-p40 antibodies, and GXP04.

12. The method of claim 1, wherein the inhibitor is a resolvin selected from the group consisting of:
Resolvin E1 ((5S, 12R, 18R)-5, 12, 18-trihydroxyicosa-6, 8, 10, 14, 16-pentaenoic acid), enantiomers and racemates thereof: 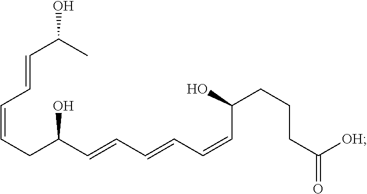 [SMILES: C[C@@H](O)/C=C/C=C\C[C@@H](O)/C=C/C=C/C=C\[C@@H](O)CCCC(=O)O]
Resolvin E2 ((5S, 18R)-5, 18-dihydroxyicosa-6, 8, 11, 14, 16-pentaenoic acid), enantiomers and racemates thereof: 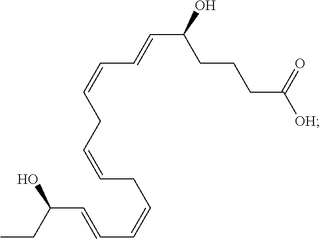 [SMILES: CC[C@@H](O)/C=C/C=C\C/C=C\C/C=C\C=C\[C@@H](O)CCCC(=O)O]
RX-10045;
Resolvin D1 ((7S, 8R, 17R)-7, 8, 17-trihydroxydocosa-4, 9, 11, 13, 15, 19-hexaenoic acid), enantiomers and racemates thereof: 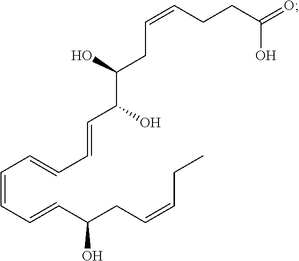 [SMILES: CC/C=C\C[C@@H](O)/C=C/C=C\C=C\C=C\[C@@H](O)[C@@H](O)C/C=C\CCC(=O)O]
Resolvin D2 ((7S, 17S)-7, 16, 17-trihydroxydocosa-4, 8, 10, 12, 14, 19-hexaenoic acid), enantiomers and racemates thereof: 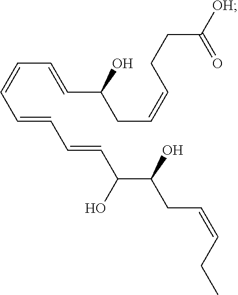 [SMILES: CC/C=C\C[C@H](O)C(O)/C=C/C=C/C=C\C=C\[C@@H](O)C/C=C\CCC(=O)O]
Resolvin D3 ((4S, 17S)-4, 11, 17-trihydroxydocosa-5, 7, 9, 13, 15, 19-hexaenoic acid), enantiomers and racemates thereof: 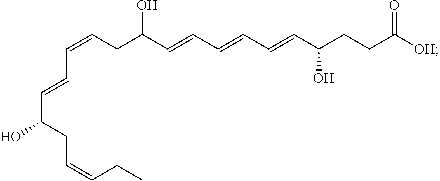 [SMILES: CC/C=C\C[C@H](O)/C=C/C=C\CC(O)/C=C/C=C/C=C/[C@@H](O)CCC(=O)O]
Resolvin D4 ((4S, 17S)-4, 5, 17-trihydroxydocosa-6, 8, 10, 13, 15, 19-hexaenoic acid), enantiomers and racemates thereof: 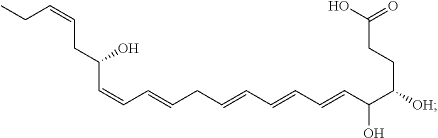 [SMILES: CC/C=C\C[C@H](O)/C=C\C=C\C/C=C/C=C/C=C/C(O)[C@@H](O)CCC(=O)O]
Resolvin D5 ((7S, 17S)-7, 17-dihydroxydocosa-5, 8, 10, 13, 15, 19-hexaenoic acid), enantiomers and racemates thereof: 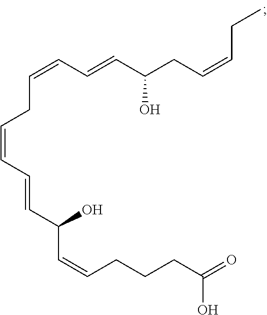 [SMILES: CC/C=C\C[C@H](O)/C=C/C=C\C/C=C\C=C\[C@@H](O)/C=C\CCCC(=O)O]
and
Resolvin D6 ((4S, 17S)-4, 17-dihydroxydocosa-5,7,10,13,15,19-hexaenoic acid), enantiomers and racemates thereof:

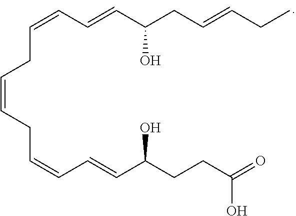 [SMILES: CC/C=C/C[C@H](O)/C=C/C=C\C/C=C\C/C=C\C=C\[C@@H](O)CCC(=O)O]

13. The method of claim 1, wherein the inhibitor is a peptide of 25 amino acid residues or fewer in length, comprising an amino acid sequence selected from the group consisting of the amino acid sequences set forth as: SEQ ID NOs 1-28.

14. The method of claim 13, wherein at least one of the amino acid residues is a D-amino acid residue.

15. A method for identifying a compound suitable for the prevention or treatment of Alzheimer's disease, comprising:
incubating cells in the presence of IL-12 and/or IL-23, and in the presence of the compound, wherein the cells comprise a receptor for IL-12 and/or a receptor for IL-23, and wherein the cells exhibit a detectable response to interaction of IL-12 with the IL-12-receptor and/or interaction of IL-23 with IL-23-receptor;
measuring the detectable response;
comparing the response in the presence of the compound to a response in a control assay, wherein a decrease in the detectable response in the cells in comparison to the response in the control assay identifies a compound suitable for the prevention or treatment of Alzheimer's disease.


SEQUENCE LISTING (28 sequences)

SEQ ID NO: 1 - protein, 8 aa, Homo sapiens
TEEEQQYL

SEQ ID NO: 2 - protein, 8 aa, Homo sapiens
TEEAQQYL

SEQ ID NO: 3 - protein, 8 aa, Homo sapiens
TAAEQQYL

SEQ ID NO: 4 - protein, 8 aa, Homo sapiens
TAAEQQYL

SEQ ID NO: 5 - protein, 8 aa, Homo sapiens
TAAAQQYL

SEQ ID NO: 6 - protein, 7 aa, Homo sapiens
EEEQQYL

SEQ ID NO: 7 - protein, 6 aa, Homo sapiens
EEQQYL

SEQ ID NO: 8 - protein, 5 aa, Homo sapiens
EQQYL

SEQ ID NO: 9 - protein, 7 aa, Homo sapiens
AEEQQYL

SEQ ID NO: 10 - protein, 8 aa, Homo sapiens
TEEEQQYL

SEQ ID NO: 11 - protein, 6 aa, Homo sapiens
TEEEQQ

SEQ ID NO: 12 - protein, 5 aa, Homo sapiens
TEEEQ

SEQ ID NO: 13 - protein, 4 aa, Homo sapiens
TEEE

SEQ ID NO: 14 - protein, 8 aa, Homo sapiens
TEEEQAYL

SEQ ID NO: 15 - protein, 8 aa, Homo sapiens
TEEEAAYL

SEQ ID NO: 16 - protein, 9 aa, Homo sapiens
MEESKQLQL

SEQ ID NO: 17 - protein, 9 aa, Homo sapiens
MAESKQLQL

SEQ ID NO: 18 - protein, 9 aa, Homo sapiens
MAASKQLQL

SEQ ID NO: 19 - protein, 7 aa, Homo sapiens
ESKQLQL

SEQ ID NO: 20 - protein, 9 aa, Homo sapiens
MEESKQLQI

SEQ ID NO: 21 - protein, 7 aa, Homo sapiens
MEESKQL

SEQ ID NO: 22 - protein, 6 aa, Homo sapiens
MEESKQ

SEQ ID NO: 23 - protein, 9 aa, Homo sapiens
MEESQQLQI

SEQ ID NO: 24 - protein, 8 aa, Homo sapiens
EESKQLQL

SEQ ID NO: 25 - protein, 24 aa, Homo sapiens
VQAANALGMEESKQLQLHLDDLVL

SEQ ID NO: 26 - protein, 24 aa, Homo sapiens
LVLDDLHLQLQKSEEMGLANAAQV

SEQ ID NO: 27 - protein, 8 aa, Homo sapiens
LPDEVTCV

SEQ ID NO: 28 - protein, 8 aa, Homo sapiens
KKYLVWVQ